(19) United States
(12) Patent Application Publication
(10) Pub. No.: US 2024/0263229 A1
(43) Pub. Date: Aug. 8, 2024

(54) HYBRID CLUSTERING

(71) Applicant: Illumina, Inc., San Diego, CA (US)

(72) Inventors: Xiaoyu Ma, San Diego, CA (US); Mathieu Lessard-Viger, San Diego, CA (US); Jeffrey Fisher, San Diego, CA (US); Jonathan Boutell, Bishops Stortford (GB)

(73) Assignee: Illumina, Inc., San Diego, CA (US)

(21) Appl. No.: 18/566,360

(22) PCT Filed: Dec. 15, 2022
(86) PCT No.: PCT/US2022/053005

§ 371 Date: Dec. 1, 2023

Related U.S. Application Data
(60) Provisional application No. 63/290,183, filed on Dec. 16, 2021.

(51) Int. Cl. C12Q 1/6874; C12N 15/10; C12Q 1/42; C12Q 1/6834; C12Q 1/6844

(52) U.S. Cl. CPC C12Q 1/6874; C12N 15/1068; C12Q 1/42; C12Q 1/6834; C12Q 1/6844; G01N 2333/916

(57) ABSTRACT

The present disclosure is generally directed to strategies for template capture and amplification during sequencing.

### CROSS-REFERENCE TO RELATED APPLICATION

This application claims the benefit of U.S. Provisional Patent Application No. 63/290,183, filed Dec. 16, 2021 and entitled “Hybrid Clustering,” the entire contents of which are incorporated by reference herein.

### FIELD

The present disclosure is generally directed to strategies for template capture and amplification during sequencing.

### BACKGROUND

The detection of analytes such as nucleic acid sequences that are present in a biological sample has been used as a method for identifying and classifying microorganisms, diagnosing infectious diseases, detecting and characterizing genetic abnormalities, identifying genetic changes associated with cancer, studying genetic susceptibility to disease, and measuring response to various types of treatment. A common technique for detecting analytes such as nucleic acid sequences in a biological sample is nucleic acid sequencing.

Advances in the study of biological molecules have been led, in part, by improvement in technologies used to characterise the molecules or their biological reactions. In particular, the study of the nucleic acids DNA and RNA has benefited from developing technologies used for sequence analysis.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing multiple extension reactions using a DNA polymerase to successively incorporate labelled nucleotides to a template strand. In such a “sequencing by synthesis” reaction a new nucleotide strand base-paired to the template strand is built up in the 5′ to 3′ direction by successive incorporation of individual nucleotides complementary to the template strand.

### SUMMARY

According to a first aspect of the disclosure, there is provided a method of amplifying a nucleic acid template, wherein the method comprises:
a. applying a nucleic acid template library in solution to a solid support; wherein the template library comprises a plurality of template strands, wherein each template strand comprises a first or second 5′ primer-binding sequence and a first or second 3′ primer binding sequence; and wherein the solid support has immobilised thereon a plurality of lawn primer sequences complementary to the 3′ primer-binding sequence;
b. hybridising the first or second 3′ primer binding sequence of the single stranded template strand to a first lawn primer;
c. carrying out an extension reaction to extend the lawn primer to generate a first immobilised strand complementary to the template strand, wherein the immobilised strand comprises a 3′ primer binding sequence;
d. displacing the template strand from the first immobilised strand and hybridising the single stranded template strand to a second lawn primer to provide said first single-stranded immobilised strand complementary to the template strand and a template strand hybridised to a second lawn primer;
e. providing a plurality of primers in solution, wherein the primers in solution are substantially complementary to the first or second 3′ primer binding sequences and hybridise to the 3′ end of the immobilised strand;
f. carrying out an extension reaction to extend the second lawn primer to generate a further immobilised stand, and the solution primer of step (e) to generate and a further template strand; and optionally
g. repeating steps (d) to (f) to produce a cluster of immobilised and template strands.

The method improves and/or addresses limitations of current amplification strategies, particularly those strategies that use bridging for amplification during cluster generation. Advantageously, it has been found that using both lawn and free solution primers for DNA amplification may result in less steric hindrance and a higher amplification flexibility. Furthermore, using primers that can only hybridize and extend, with no invasion capability, may minimise or prevent the formation of duplicates, which are detrimental amplification efficiency and downstream sequencing performance.

According to a further aspect of the disclosure, there is provided a method of sequencing a nucleic acid sequence, wherein the method comprises:
a. applying a nucleic acid template library in solution to a solid support; wherein the template library comprises a plurality of template strands, wherein each template strand comprises a first or second 5′ primer-binding sequence and a first or second 3′ primer binding sequence; and wherein the solid support has immobilised thereon a plurality of lawn primer sequences complementary to the 3′ primer-binding sequence; and a plurality of dormant lawn primers substantially complementary to the 3′ first or second primer-binding sequence, wherein the dormant lawn primers are blocked at the 3′end, and wherein the lawn and dormant lawn primers bind to different 3′-primer binding sequences;
b. hybridising the first or second 3′ primer binding sequence of the single stranded template strand to a first lawn primer;
c. carrying out an extension reaction to extend the lawn primer to generate a first immobilised strand complementary to the template strand, wherein the immobilised strand comprises a 3′ primer binding sequence;
d. displacing the template strand from the first immobilised strand and hybridising the single stranded template strand to a second lawn primer to provide said first single-stranded immobilised strand complementary to the template strand and a template strand hybridised to a second lawn primer;
e. providing a plurality of primers in solution, wherein the primers in solution are substantially complementary to the first or second 3′ primer binding sequences and hybridise to the 3′ end of the immobilised strand;
f. carrying out an extension reaction to extend the second lawn primer to generate a further immobilised stand, and the solution primer of step (e) to generate and a further template strand; and optionally
g. repeating steps (d) to (f) to produce a cluster of immobilised and template strands;
   and additionally,
h. selectively removing the non-immobilised template strands;
i. carrying out a first sequencing read to determine the sequence of a region of the immobilised strand; preferably by a sequencing-by-synthesis technique or by a sequencing-by ligation technique;
j. selectively removing the sequencing product;
k. removing the blocking group from the dormant primers to allow hybridisation of the 3′ end of the immobilised strand to the unblocked primer;
l. carrying out an extension reaction to extend the unblocked primer using the immobilised strand as a template;
m. selectively removing the immobilised first sequencing read strand; and
n. carrying out a second sequencing read to determine the sequence of a region of the immobilised strand; preferably by a sequencing-by-synthesis technique or by a sequencing-by ligation technique.

This method may significantly shorten paired-end read re-synthesis time.

According to a yet further aspect of the disclosure, there is provided a method of sequencing a target nucleic acid sequence, wherein the method comprises:
a. providing a solid support having immobilised thereon a cluster of first immobilised nucleic acid strands including said target nucleic acid sequence, wherein the solid support has a plurality of dormant lawn primers, wherein the dormant lawn primers are blocked at the 3′end;
b. carrying out a first sequencing read to determine the sequence of a region of the first immobilised strands; preferably by a sequencing-by-synthesis technique or by a sequencing-by ligation technique;
c. removing the blocking group from the dormant primers to allow hybridisation of a 3′ end of the first immobilised strand to the unblocked primer;
d. carrying out an extension reaction to extend the unblocked primer using the immobilised strand as a template to generate a cluster of second immobilised nucleic acid strands;
e. carrying out a second sequencing read to determine the sequence of a region of the second immobilised strand; preferably by a sequencing-by-synthesis technique or by a sequencing-by ligation technique;
wherein determining the first and second sequences achieves pairwise sequencing of said target nucleic acid sequence.

Again, this method may significantly shorten paired-end read re-synthesis timecycles. The methods of the present disclosure can be advantageously used in pairwise sequencing of target nucleic acid sequences.

According to a yet further aspect of the disclosure, there is provided a solution-phase primer comprising or consisting of a nucleic acid sequence as defined in SEQ ID NO: 5, 6 or 7 or a variant thereof.

The solution-phase primers of the present disclosure may be useful in the methods of the present disclosure and may advantageously minimise or prevent duplication.

According to a yet further aspect of the disclosure, there is provided a re-synthesis primer, the primer comprising a nucleic acid sequence selected from SEQ ID NO: 9, 10 or 11 or a variant thereof, wherein the primer is blocked at the 3′ end, wherein the block prevents extension of the primer until the block is removed.

According to a yet further aspect of the disclosure, there is provided a solid support for use in sequencing, wherein the support comprises a plurality of lawn primers immobilised thereon and a plurality of dormant lawn primers immobilised thereon, wherein the dormant lawn primers comprise a blocking 3′ group that prevents extension until removed.

The re-synthesis primers according to the present disclosure advantageously prevent bridged amplification during initial cluster generation which may minimise or avoid amplification propagating into adjacent wells. It may also advantageously provide pristine primers to be available during a second sequencing read, as the primers have not previously be used during bridge amplification.

According to a yet further aspect of the disclosure, there is provided a hybridisation buffer, wherein the hybridisation buffer comprises a denaturation agent and at least one solution-phase primer of the disclosure.

According to a yet further aspect of the disclosure, there is provided a buffer, wherein the buffer comprises at least one solution-phase primer of the disclosure.

### BRIEF DESCRIPTION OF DRAWINGS

FIG. 1A shows library size corresponding to the number of base pairs. FIG. 1B shows that as the interstitial space between nanowells is reduced to levels that may be smaller than the size of the library elements, amplification can propagate into adjacent wells. FIG. 1C shows DNA clusters on lawn of flow cell with different patterned interstitial spaces.

FIGS. 2A-2D. scheme of the hybrid clustering: FIG. 2A shows primer P7 grafted on the surface. Both unblocked Lawn and free-solution phase primers participate in the exponential clustering step. The lawn primers enable the “walking” and extension of the template, and the solution-phase primer can only hybridise/extend, ascribing to less RPA invasion efficiency of the shorter solution-phase primer. FIG. 2B also shows primer P7 but also shorter blocked P5 (blocked with phosphate at 3′ end). The shorter blocked P5 primers can be deprotected prior to PE turn, where the usage of shorter stumps can avoid slowing down of the amplification mix (for example ExAmp, which is an amplification mix comprising non-thermostable strand displacement polymerase BSU).

FIG. 2C shows strands amplification as measured by the intensity of intercalating dye over time, and illustrates good clustering performance. FIG. 2D shows relation between P7 lawn primer densities and resulting sequencing intensity and % pass filter (PF). Due to lower steric hindrance embodiments herein higher sequencing intensities for equivalent primer densities with non-bridging clustering.

FIG. 3A show an investigation of hybrid clustering using free solution primers at different concentrations. The curve is the real-time EvaGreen intensity. FIG. 3B shows fluorescence intensity of hybridized dye-labelled sequencing primer, which is applied to represent the final cluster intensity.

FIG. 4. Kinetics comparison between as-designed hybrid (free solution primers at 5 μM) and current clustering.

FIG. 5. Origin of the sequencing duplicates in both Illumina clustering strategy (labelled with purple dot) and as-designed hybrid clustering methodology (labelled with check marks).

FIGS. 6A-6B. Scheme of behaviour of “smart” solution primers: they can only hybridise/extend, but not invade.

FIGS. 7A-7D. Scheme of the solution-based invasion (FIG. 7A) and hybridise/extension (FIG. 7C) assay, where BHQ represents fluorophore, and FAM represents fluorescence quencher. In both assays, fluorescence is initially quenched, then with the tested solution P5 either invasion/extension (FIG. 7A) or hyb/extension (FIG. 7C), the quencher modified complementary strand would be kicked off, resulting in the turn-on of the fluorescence intensity. (FIG. 7B) and (FIG. 7D) show the corresponding result of invasion and hyb/extension efficiency of solution P5 at different lengths (15 bases, 13 bases, 10 bases, and control 29 bases), respectively. The experiment demonstrates the ability to use primer length to tune the invasion function, without negatively affecting the hyb/extension function.

FIG. 8A shows percent of duplicates formed where short solution and full-length P5 primers are used. FIG. 8B shows a sequencing matrix comparing P90 (intensity value from each cluster), PF and duplicates between normal bridging clustering strategy and hybrid clustering methodology. Orange and blue bars represent the conditions of normal clustering and hybrid clustering, respectively. P5-13 represents solution primer of 13 bp′ P5. P5-C represents the normal P5 with 29 bp.

FIG. 9 shows a scheme of the PE re-synthesis.

FIG. 10A shows a comparison of read 2 intensity using hybrid clustering after different re-synthesis cycles (blue bar), where normal Illumina clustering strategy is employed as the control (orange bar). FIG. 10B shows sequencing intensity of a PE run (36 by 36 cycles) using hybrid clustering under 1 cycle of re-synthesis.

FIGS. 11A-11C show the signal intensity for fast paired end turn using ExAmp with one push for 5 min. FIG. 11A shows the standard conditions for ExAmp as a control. FIG. 11B shows non-bridging clustering using the 10 base pair, blocked, short P5 primer (BsP5) FIG. 11C shows non-bridging clustering using the 13 base pair, blocked, short P5 primer (BsP5).

FIGS. 12A-12B shows the ratio between lawn-P7 primer binding sequences and the dormant-P5s affects the R1 and R2 intensity. A higher concentration of BsP5 results in better PE turn but lower R1 intensity (P7: 1.1 μM; ExAmp: Ras6T; Library: N450 at 200 pM).

FIG. 13 shows that the decrease in R1 intensity when using BsP5 is probably due to unwanted annealing with the templates. However, further shortening of the length of BsP5 can be used to further lower the Tm and inhibit unwanted annealing.

FIG. 14 is a schematic of generation of a single-stranded library from a double-stranded template library.

### DETAILED DESCRIPTION

The following features apply to all aspects of the present disclosure.

The present disclosure can be used in sequencing, for example pairwise sequencing. Methodology applicable to the present disclosure have been described in WO 08/041002, WO 07/052006, WO 98/44151, WO 00/18957, WO 02/06456, WO 07/107710, WO05/068656, U.S. Ser. No. 13/661,524 and US 2012/0316086, the contents of which are herein incorporated by reference. Further information can be found in US 20060024681, US 200602926U, WO 06110855, WO 06135342, WO 03074734, WO07010252, WO 07091077, WO 00179553 and WO 98/44152, the entire contents of each which are incorporated by reference herein.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts.” Alternatively “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adaptor sequences.

As used herein an “adapter” sequence comprises a short sequence-specific oligonucleotide that is ligated to the 5′ and 3′ ends of each DNA (or RNA) fragment in a sequencing library as part of library preparation. The adaptor sequence may further comprise non-peptide linkers.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

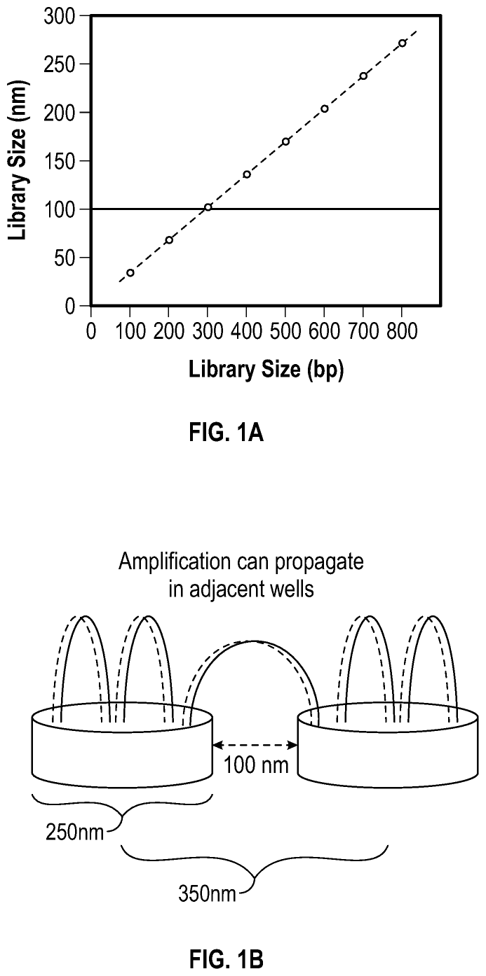

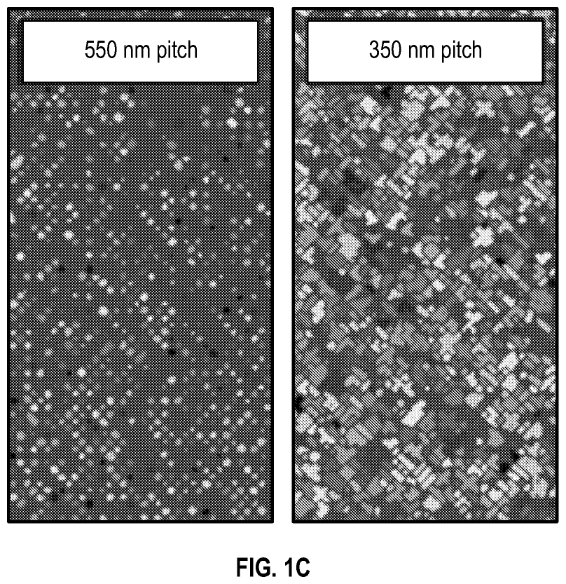

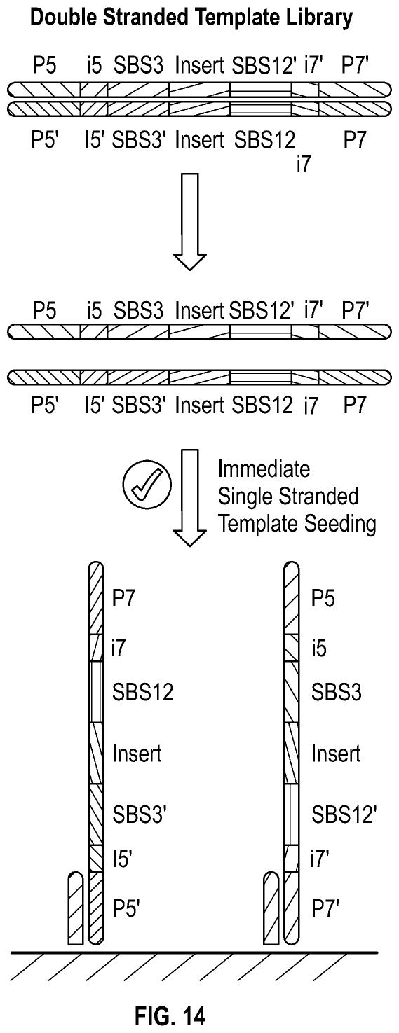

An example of a typical single-stranded nucleic acid template is shown in FIG. 14. In one embodiment, the template comprises, in the 5′ to 3′ direction, a first primer-binding sequence (e.g. P5), an index sequence (e.g. i5), a first sequencing binding site (e.g. SBS3), an insert, a second sequencing binding site (e.g. SBS12′), a second index sequence (e.g. i7′) and a second primer-binding sequence (e.g. P7′). In another embodiment, the template comprises, in the 3′ to 5′ direction, a first primer-binding site (e.g. P5′, which is complementary to P5), an index sequence (e.g. i5′, which is complementary to 15), a first sequencing binding site (e.g. SBS3′ which is complementary to SBS3), an insert, a second sequencing binding site (e.g. SBS12, which is complementary to SBS12), a second index sequence (e.g. i7, which is complementary to 17) and a second primer-binding sequence (e.g. P7, which is complementary to P7′). Either template is referred to herein as a “template strand” or “a single stranded template”. Both template strands annealed together as shown in FIGS. 1A-1C, is referred to herein as “a double stranded template”. The combination of a primer-binding sequence, an index sequence and a sequencing binding site is referred to herein as an adaptor sequence, and a single insert is flanked by a 5′ adaptor sequence and a 3′ adaptor sequence. The first primer-binding sequence may also comprise a sequencing primer for the index read (15).

In one embodiment, the P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein “'” denotes the complementary strand.

The primer-binding sequences in the adaptor which permit hybridisation to amplification primers will typically be around 20-40 nucleotides in length, although, in embodiments, the disclosure is not limited to sequences of this length. The precise identity of the amplification primers, and hence the cognate sequences in the adaptors, are generally not material to the disclosure, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art. “Primer-binding sequences” may also be referred to as “clustering sequences” “clustering primers” or “cluster primers” in the present disclosure, and such terms may be used interchangeably.

The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analyzed in a single run, without drastically increasing run cost or run time. Examples of tag sequences are found in WO05068656, the entire contents of which are incorporated by reference herein. The tag can be read at the end of the first read, or equally at the end of the second read. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S. 60/899,221, the entire contents of which are incorporated by reference herein. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The DNA polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation, such as NaOH or formamide, is used. Suitable denaturation agents include: acidic nucleic acid denaturants such as acetic acid, HCl, or nitric acid; basic nucleic acid denaturants such as NaOH; or other nucleic acid denaturants such as DMSO, formamide, betaine, guanidine, sodium salicylate, propylene glycol or urea. Preferred denaturation agents are formamide and NaOH, preferably formamide.

Following denaturation, a single-stranded template library is in one embodiment contacted in free solution onto a solid support comprising surface capture moieties (for example P5 and/or P7 primers). This solid support is typically a flowcell, although in alternative embodiments, seeding and clustering can be conducted off-flowcell using, for example, microbeads or the like.

As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fibre bundles, and polymers. A particularly useful material is glass. Other suitable substrate materials may include polymeric materials, plastics, silicon, quartz (fused silica), boro float glass, silica, silica-based materials, carbon, metals including gold, an optical fibre or optical fibre bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g. being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture. Further examples of materials that can be used in the structured substrates or methods of the present disclosure are described in U.S. Ser. No. 13/661,524 and US Pat. App. Pub. No. 2012/0316086 A1, the entire contents of each are incorporated by reference herein.

The disclosure may make use of solid supports comprised of a substrate or matrix (e.g. glass slides, polymer beads etc) which has been “functionalised”, for example by application of a layer or coating of an intermediate material comprising reactive groups which permit covalent attachment to biomolecules, such as polynucleotides. Examples of such supports include, but are not limited to, a substrate such as glass. In such embodiments, the biomolecules (e.g. polynucleotides) may be directly covalently attached to the intermediate material but the intermediate material may itself be non-covalently attached to the substrate or matrix (e.g. the glass substrate). The term “covalent attachment to a solid support” is to be interpreted accordingly as encompassing this type of arrangement. Alternatively, the substrate such as glass may be treated to permit direct covalent attachment of a biomolecule; for example, glass may be treated with hydrochloric acid, thus exposing the hydroxyl groups of the glass, and phosphite-triester chemistry used to directly attach a nucleotide to the glass via a covalent bond between the hydroxyl group of the glass and the phosphate group of the nucleotide.

In other embodiments, the solid support may be “functionalised” by application of a layer or coating of an intermediate material comprising groups that permit non-covalent attachment to biomolecules. In such embodiments, the groups on the solid support may form one or more of ionic bonds, hydrogen bonds, hydrophobic interactions, π-π interactions, van der Waals interactions and host-guest interactions, to a corresponding group on the biomolecules (e.g. polynucleotides). The interactions formed between the group on the solid support and the corresponding group on the biomolecules may be configured to cause immobilisation or attachment under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

In other embodiments, the solid support may be “functionalised” by application of an intermediate material comprising groups that permit attachment via metal-coordination bonds to biomolecules. In such embodiments, the groups on the solid support may include ligands (e.g. metal-coordination groups), which are able to bind with a metal moiety on the biomolecule. Alternatively, or in addition, the groups on the solid support may include metal moieties, which are able to bind with a ligand on the biomolecule. The metal-coordination interactions formed between the ligand and the metal moiety may be configured to cause immobilisation or attachment of the biomolecule under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

When referring to immobilisation or attachment of molecules (e.g. nucleic acids) to a solid support, the terms “immobilised” and “attached” are used interchangeably herein and both terms are intended to encompass direct or indirect, covalent or non-covalent attachment, unless indicated otherwise, either explicitly or by context. In certain embodiments of the disclosure, covalent attachment may be preferred; in other embodiments, attachment using non-covalent interactions may be preferred; in yet other embodiments, attachment using metal-coordination bonds may be preferred. However, in general the molecules (e.g. nucleic acids) remain immobilised or attached to the support under the conditions in which it is intended to use the support, for example in applications requiring nucleic acid amplification and/or sequencing. When referring to attachment of nucleic acids to other nucleic acids, then the terms “immobilised” and “hybridised” are used herein, and generally refer to hydrogen bonding between complementary nucleic acids.

If the amplification is performed on beads, either with a single or multiple extendable primers, the beads may be analysed in solution, in individual wells of a microtitre or picotitre plate, immobilised in individual wells, for example in a fibre optic type device, or immobilised as an array on a solid support. The solid support may be a planar surface, for example a microscope slide, wherein the beads are deposited randomly and held in place with a film of polymer, for example agarose or acrylamide.

As described above, once a library comprising template nucleotide strands has been prepared, the templates are seeded onto a solid support and then amplified to generate a cluster of single template molecules.

By way of brief example, following attachment of the P5 and P7 primers, the solid support may be contacted with the template to be amplified under conditions which permit hybridisation (or annealing—such terms may be used interchangeably) between the template and the immobilised primers (also referred to herein as “lawn primers”). The template is usually added in free solution under suitable hybridisation conditions, which will be apparent to the skilled reader. Typically, hybridisation conditions are, for example, 5×SSC at 40° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which in some methods is capable of bridging to the second primer molecule immobilised on the solid support and binding. In this method, further rounds of amplification (analogous to a standard PCR reaction) lead to the formation of clusters or colonies of template molecules bound to the solid support. Thus, in this example, solid-phase amplification by either the method analogous to that of WO 98/44151 or that of WO 00/18957 (the contents of which are incorporated herein in their entirety by reference) will result in production of a clustered array comprised of colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582, the entire contents of which are incorporated by reference herein. The method may also involve a number of rounds of invasion by a competing immobilised primer (or lawn primer) and strand displacement of the template to the competing primer. Further information on amplification can be found in WO0206456 and WO07107710, the entire contents of each of which are incorporated by reference herein. Through such approaches, a cluster of single template molecules is formed.

To facilitate sequencing, it is preferable if one of the strands is removed from the surface to allow efficient hybridisation of a sequencing primer to the remaining immobilised strand. Suitable methods for linearisation are described in more detail in application number WO07010251, the entire contents of which are incorporated by reference herein.

Sequence data can be obtained from both ends of a template duplex by obtaining a sequence read from one strand of the template from a primer in solution, copying the strand using immobilised primers, releasing the first strand and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′-end. If the first strand is removed from the surface, the sequence of the second strand can be read. This gives a sequence read from both ends of the original fragment. The process whereby the strand is regenerated after the first read is known as “Paired-end resynthesis”. The typical steps of pairwise sequencing are known and have been described in WO 2008/041002, the entire contents of which are incorporated by reference herein.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB/2007/001770, the entire contents of which are incorporated by reference herein. Alternatively, a separate reaction may be carried out containing each of the modified nucleotides added individually.

The modified nucleotides may carry a label to facilitate their detection. In a particular embodiment, the label is a fluorescent label. Each nucleotide type may carry a different fluorescent label. However the detectable label need not be a fluorescent label. Any label can be used which allows the detection of the incorporation of the nucleotide into the DNA sequence. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the entire contents of which are incorporated by reference herein.

Alternative methods of sequencing include sequencing by ligation, for example as described in U.S. Pat. No. 6,306,597 or WO06084132, the entire contents of each of which are incorporated by reference herein.

However, current bridge-based clustering methods may limit the density of nanowells that can be used on any solid support. As shown in FIGS. 1A-1C, as the nanowell density increases, it becomes possible for the products of cluster amplification propagating into adjacent wells. This is particularly problematic where the interstitial space or pitch between nanowells is small, and in particular where the space is smaller than the size of the library elements (for example, less than 550 nm, e.g. 350 nm). This is shown in FIGS. 1A and 1B.

The disclosure solves this problem by clustering without bridging. This may be referred to as “hybrid clustering”. Clustering without bridging is achieved in this disclosure by the use of free solution primers, in addition to immobilised (or lawn primers). In an embodiment, these are either free solution P5 or free solution P7 primers, and replace the use of the respective P5 and P7 lawn primers.

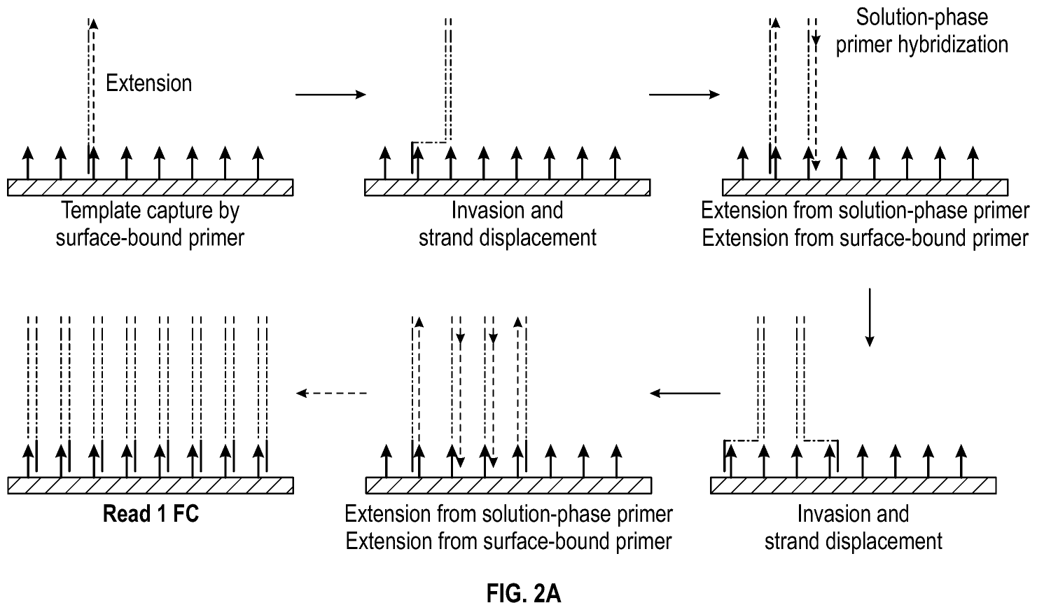

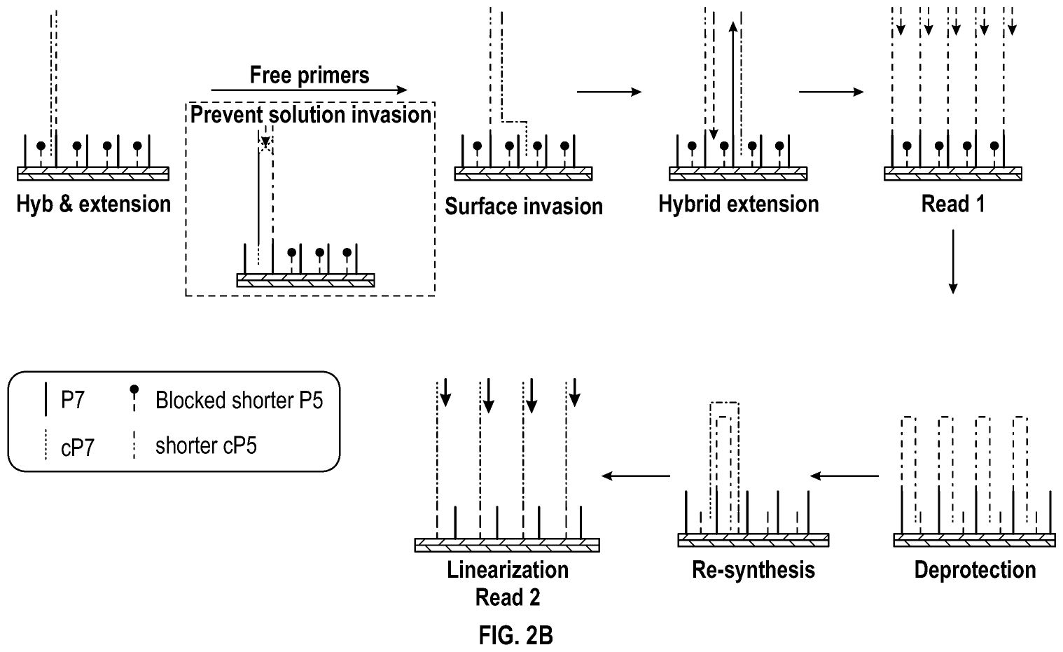

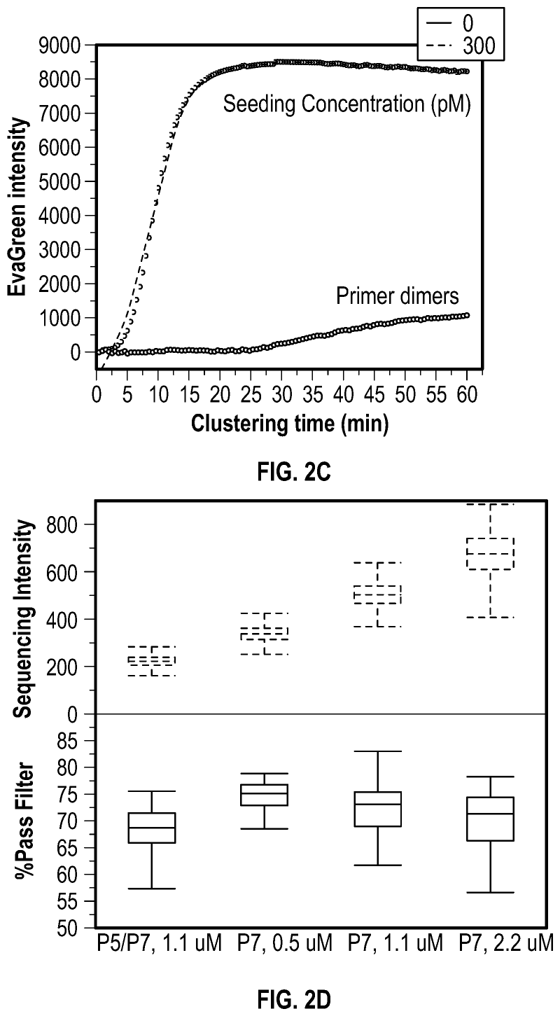

One embodiment of the hybrid clustering method of the disclosure is shown in FIGS. 2A-2D. In the first step, a single stranded template library is contacted with a solid support on which the amplification primers (e.g. P5 or P7) are immobilised (these are referred to herein as “lawn primers”) under conditions that allow hybridisation between the template and the primers. Typically, hybridisation conditions are, for example, 5×SSC at 38° C. Solid-phase amplification can then proceed. The first step of amplification is a primer extension step in which nucleotides are added to the 3′ end of the lawn primer using the template to produce a fully extended complementary strand (i.e. “the complement”). After formation of a double strand of DNA, there follows a step of surface strand invasion and strand displacement, wherein the lawn primer invades the double strand of DNA and displaces the template from the now elongated first lawn primer. The result is a single-stranded extended complementary strand immobilised to the solid support and a template strand hybridised to a second lawn primer. The fully extended complementary strand will include at its 3′ end a primer-binding sequence (i.e. either P5′ or P7′). In the next step of amplification, a solution-phase primer (that is, a primer in free solution) is present. The solution phase primer hybridises to the 3′ end of the extended complementary strand (e.g. the solution phase primer is a P7 or P5 primer and binds to P7′ or P5′). Hybridisation conditions may be the same as above—e.g. 5×SSC at 38° C. Following hybridisation of the solution-phase primer, the next stage is primer extension, in which nucleotides are added to the 3′ end of the hybridised solution primer using the complementary strand as a template to produce a fully extended complementary strand. At the same time, the second lawn primer is extended (nucleotides are added to the 3′ end of the lawn primer) using the template strand to produce a further fully extended complementary strand. The steps of invasion and strand displacement and extension from both the surface i.e. lawn) and solution-phase primers are repeated until a cluster of linear template strands have generated.

Accordingly, the disclosure provides a method of amplifying a nucleic acid template, wherein the method comprises the following steps:
a. applying a nucleic acid template library in solution to a solid support; wherein the template library comprises a plurality of template strands, wherein each template strand comprises a first or second 5′ primer-binding sequence and a first or second 3′ primer binding sequence; and wherein the solid support has immobilised thereon a plurality of lawn primer sequences complementary to the 3′ primer-binding sequence;
b. hybridising the first or second 3′ primer binding sequence of the single stranded template strand to a first lawn primer;
c. carrying out an extension reaction to extend the lawn primer to generate a first immobilised strand complementary to the template strand, wherein the immobilised strand comprises a 3′ primer binding sequence;
d. displacing the template strand from the first immobilised strand and hybridising the single stranded template strand to a second lawn primer to provide said first single-stranded immobilised strand complementary to the template strand and a template strand hybridised to a second lawn primer;
e. providing a plurality of primers in solution, wherein the primers in solution are substantially complementary to the first or second 3′ primer binding sequences and hybridise to the 3′ end of the immobilised strand;
f. carrying out an extension reaction to extend the second lawn primer to generate a further immobilised stand, and the solution primer of step (e) to generate and a further template strand; and optionally
g. repeating steps (d) to (f) to produce a cluster of immobilised and template strands.

In an embodiment, steps (d) to (f) are repeated through multiple cycles in the presence of an isothermal recombinase at 38ºC for about 1 hour.

In an embodiment, in step (e), the solution containing the plurality of primers may be the same solution from step (a). In a further embodiment, the solution containing the solution primers may be a different solution. Said another way, the solution primers may be added into the system at various stages depending on the methodology used. In some embodiment, the solution primers may be added during the process whereas in other embodiments the solution primers are present at the start of the process.

Following step (i) of the recited method, the template strands may be washed off the solid support.

By “nucleic acid template library” is meant a plurality of template nucleic acid strands comprising an insert, which is the samples nucleic acid flanked by 5′ and 3′ adaptor sequences that allow amplification and sequencing of the insert. Examples of adaptor sequences are described above. Preferably the adaptor sequences comprise 5′ and 3′ primer-binding sequences. The template nucleic acid strands may be initially double-stranded as shown in FIG. 14, but are denatured prior to amplification to form a cluster and sequencing.

The term “cluster” refers to a discrete site on a solid support comprised of a plurality of identical immobilised nucleic acid strands.

By “complementary” is meant that the primer has a sequence of nucleotides that can form a double-stranded structure by matching base-pairs with the adaptor or primer sequence or part thereof. By “substantially complementary” is meant that the primer has at least 85%, 90%, 95%, 98%, 99% or 100% overall sequence identical to the complementary sequence.

The terms “hybridise” and “anneal” can be used interchangeably. In one embodiment, hybridisation occurs under 5×SSC (saline sodium citrate) at 38° C.

An extension reaction, in which nucleotides are added to the 3′ end of a primer is performed using a polymerase, such as a DNA or RNA polymerase. In one embodiment, the polymerase is a non-thermal isothermal strand displacement polymerase. Suitable non-thermostable strand displacement polymerases according to the present disclosure can be found, for example, through New England BioLabs, Inc. and include phi29, Bsu, Klenow, DNA Polymerase I (E. coli), and Therminator. A particularly preferred polymerase is Bsu.

In an embodiment, the template strands comprise either a first 3′ primer-binding sequence or a second 3′ primer binding sequence, where the sequence of the first and second primer binding sequences are different. In this embodiment, the lawn primer is substantially complementary to either the first or second 3′ primer-binding sequence and the primer added in solution (referred to herein as the solution phase primer) is substantially complementary to the first or second 3′ primer binding sequence, wherein the immobilised and solution phase primer do not bind to the same 3′ primer binding sequence. In other words, only one type of lawn primer participates in the amplification/cluster generation step.

In a preferred embodiment, each single stranded template comprise a 5′ primer-binding sequence that is either a P5 or P7 primer-binding sequence and a 3′ primer-binding sequence that is either a P5′ or P7′ primer-binding sequence. In one embodiment, the lawn primer is a P5 or P7 primer. In another embodiment, the solution phase primer is a P5 or P7 primer.

In one embodiment, the lawn primer is a P7 primer and the solution phase primer is a P5 primer. In this embodiment, the lawn primer binds to P7′ on the 3′ end of the template strand, where P7′ is substantially complementary to P7. In this embodiment, the solution-phase primer binds to P5′ on the 5′ end of the immobilised strand, where P5′ is substantially complementary to P5.

In an alternative embodiment, the lawn primer is a P5 primer and the solution phase primer is a P7 primer. In this embodiment, the lawn primer binds to P5′ on the 3′ end of the template strand, where P5′ is substantially complementary to P5. In this embodiment, the solution-phase primer binds to P7′ on the 5′ end of the immobilised strand, where P7′ is substantially complementary to P7.

In one embodiment, the sequence of P5 comprises or consists of SEQ ID NO: 1 or a variant thereof, the sequence of P5′ comprises or consists of SEQ ID NO: 3 or a variant thereof, the sequence of P7 comprises or consist of SEQ ID NO: 2 or a variant thereof and the sequence of P7′ comprises or consists of SEQ ID NO: 4 or a variant thereof.

The term “variant” as used herein with reference to any of the sequences recited herein refers to a variant nucleic acid that is substantially identical, i.e. has only some sequence variations, for example to the non-variant sequence. In one embodiment, a variant has at least 50%, 55%, 60%, 65%, 70%, 75%, 80%, 85%, 86%, 87%, 88%, 89%, 90%, 91%, 92%, 93%, 94%, 95%, 96%, 97%, 98%, or at least 99% overall sequence identity to the non-variant nucleic acid sequence.

Of course, reference to P5 and P7 could refer to different primer sequences. Any suitable primer sequence combinations are encompassed by the present disclosure. P5′ and P7′ are complementary (as defined herein) to P5 and P7.

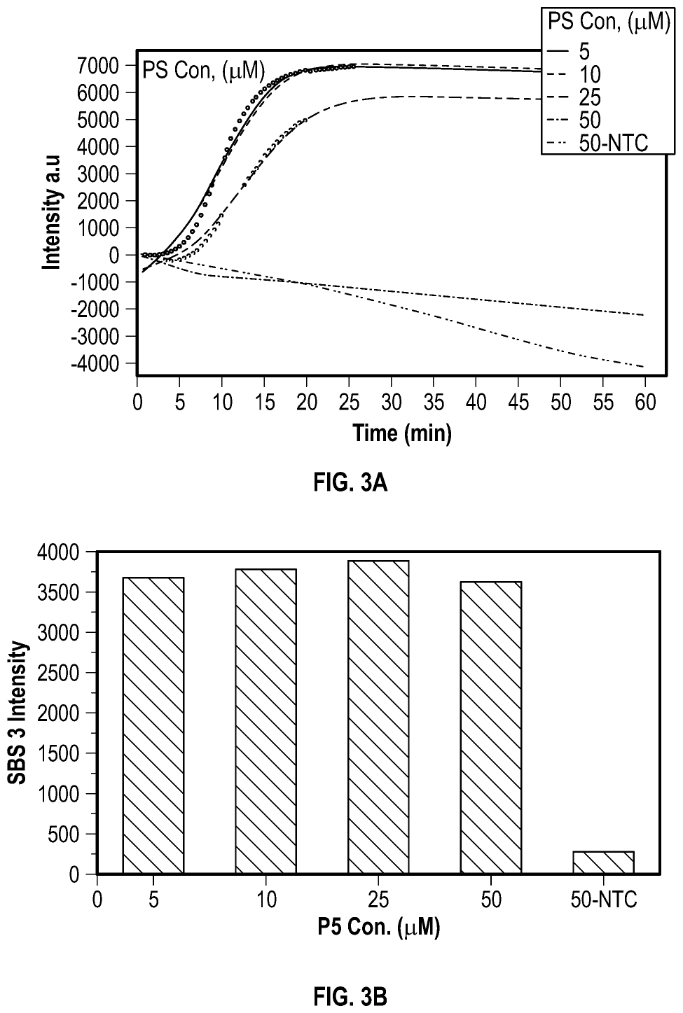

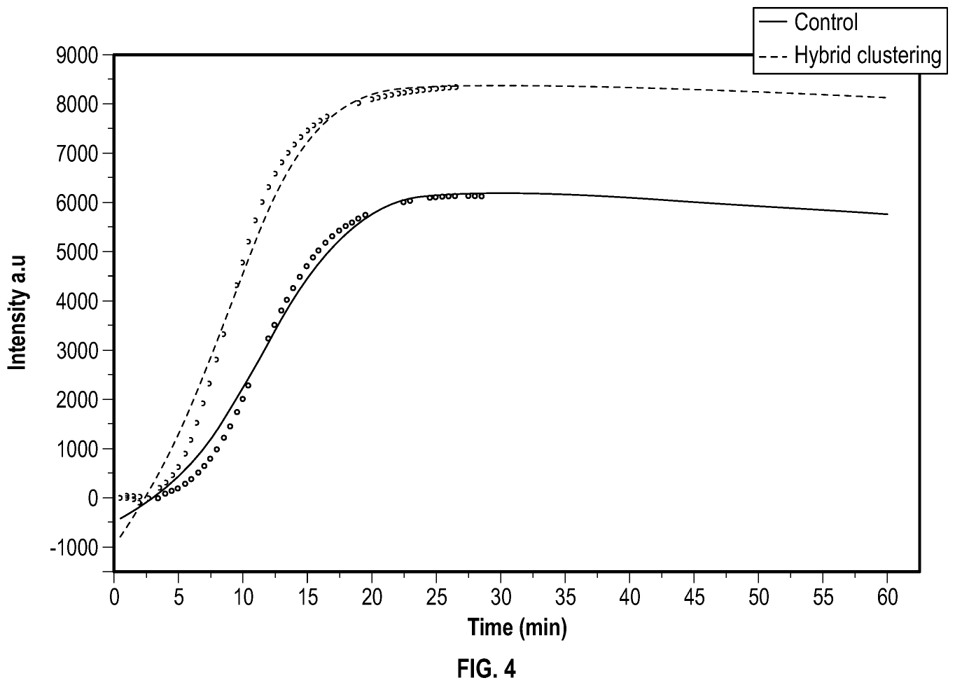

Evidence that the non-bridging method of the disclosure resulted in the formation of clusters is shown in FIGS. 3A-3B. Here, real-time cluster formation was measured using lawn P7 lawn primers immobilised at 1.1 μM and P5 solution primers ranging in concentration from 5 μM to 50 μM. In FIG. 3A, real-time clustering was measured using Evergreen intensity as a read-out of the formation of double-stranded DNA (Evagreen is a green fluorescent nucleic acid dye that is non-fluorescent by itself but becomes highly fluorescent upon binding to double-stranded DNA) (FIG. 3A). In FIG. 3B, final cluster intensity was also assessed by measuring the fluorescence intensity of hybridised dye-labelled sequencing primer. Both figures show that clusters are formed using the methods of the disclosure. Moreover, as shown in FIG. 4, the method of the disclosure leads to faster clustering kinetics and greater levels of clustering compared to bridging methods where both primers are immobilized on the surface.

The use of only one type of lawn primers combined with the use of solution primers in step (f) allows amplification of the template strand without needing a bridging step. This in turn prevents propagation of amplification into adjacent wells, resulting in less steric hindrance, reducing the pitch possible between wells and consequently leads to faster clustering. FIG. 2C shows real-time clustering kinetics curve for this method by using fluorescent intensity of the intercalating dye. FIG. 2D shows the sequencing intensity and % Pass Filter of bridging clusters (P5/P7) and non-bridging clusters with lawn P7 primers at different concentrations (0.5, μM, 1.1 μM and 2.2 μM) was compared. % PF is a measure of the ability of a nanowell to be successfully ‘read’ during sequencing. As shown in FIG. 2D, at all concentrations of lawn P7 primer tested, non-bridging clustering led to a higher % PF and a higher sequencing intensity.

Accordingly, in one embodiment, the lawn primer is grafted at a concentration in the range of 0.2 μM to 5 μM or 0.4 μM to 3 μM or 0.5 μM to 2.5 μM. In a further embodiment, the lawn primer is grafted at 0.5, μM or 1.1 μM or 2.2 μM. In a preferred embodiment, the lawn immobilised primer is grafted at 2.2 μM. The lawn primer is either a P5 or P7 primer.

In another embodiment, the solution-phase primer is used at a concentration in the range of 1 μM to 100 μM or 3 μM to 75 μM or 5 to 50 M. In a further embodiment, the solution-phase primer is used at 0.5, μM or 1.1 μM or 2.2 μM. In a preferred embodiment, solution-phase primer is used at 1 μM 5 μM, 10 μM, 25 UM or 50 μM. The solution-phase primer is either a P5 or P7 primer.

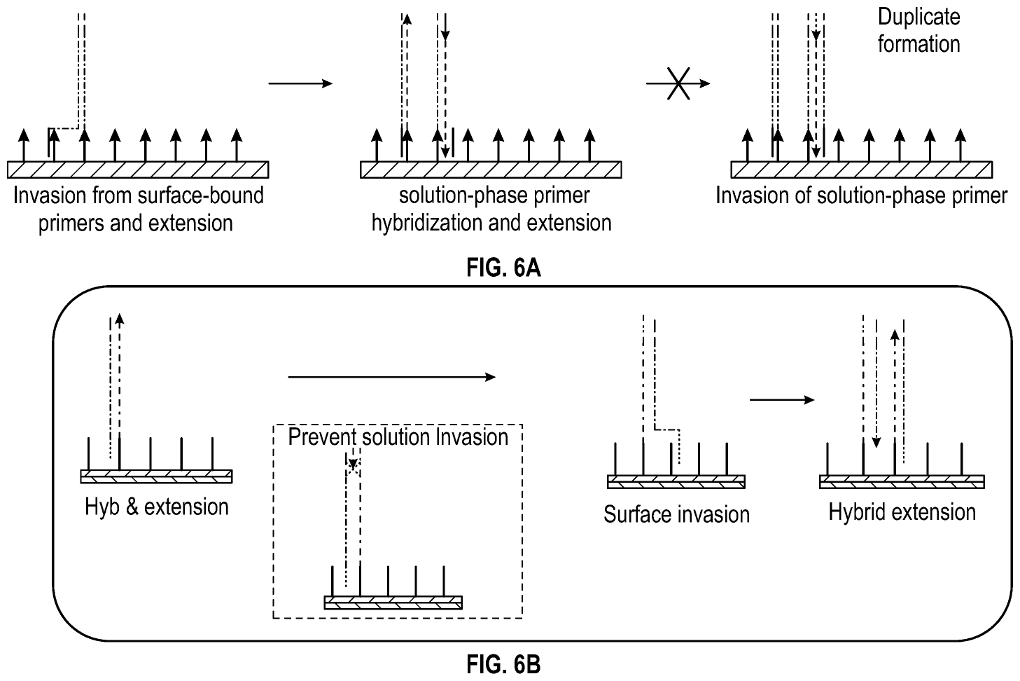

Following the step of hybridisation and extension from the solution-phase primers, it is possible for another solution-phase primer to invade the newly formed duplex and extend again the same template strand, thereby creating duplicates. This is shown in FIG. 6A. The present disclosure has identified a system whereby lawn primers can invade and extend using the bound template strand and solution-phase primers can hybridise and extend, but importantly not invade an already-formed duplex. This disclosure solves this problem using variant solution primers to those described above. These primers are referred to herein as “smart solution primers” or “shorter solution primers”. This is the first demonstration of a method that uses solution primers while also preventing unwanted duplicates.

In one embodiment, the extension reaction is carried out by recombinase polymerase amplification (RPA). RPA comprises three core enzymes—a recombinase, a single-stranded DNA binding protein (SSB) and strand-displacing polymerase. As described in Daher et al. (Rana K Daher, Gale Stewart, Maurice Boissinot, Michel G Bergeron, Recombinase Polymerase Amplification for Diagnostic Applications, Clinical Chemistry, Volume 62, Issue 7, 1 Jul. 2016). The recombinase is responsible for strand invasion by forming filaments with the primers. It has been found that preventing the formation of recombinase-primer filaments reduces the formation of duplicates. In one embodiment, this can be achieved by reducing the length of the primers. In particular, without wishing to be bound by theory, shortening the length of the primers may avoid filament formation between the recombinase and the primers, thereby leading to reduced or no strand displacement. In this manner a solution primer is achieved that is capable of hybridisation and elongation but not invasion, thereby preventing or reducing the formation of duplicates. This is shown in FIG. 6B.

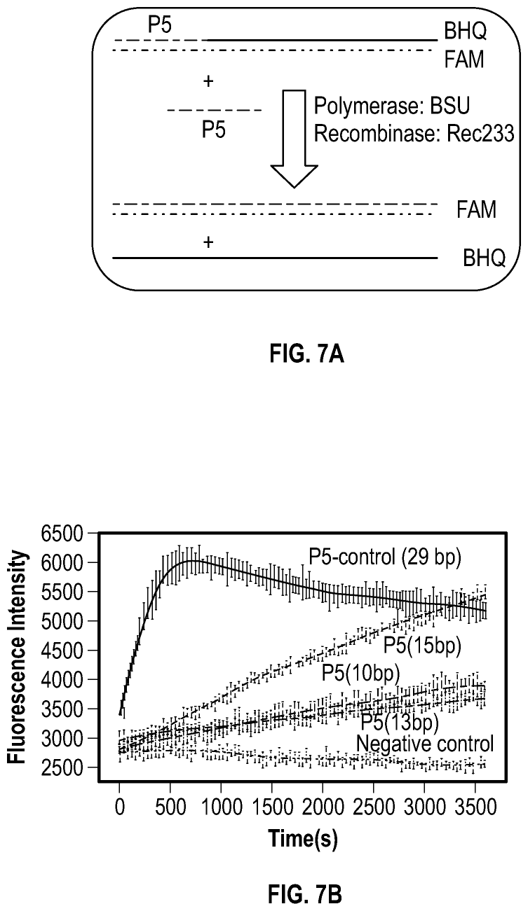

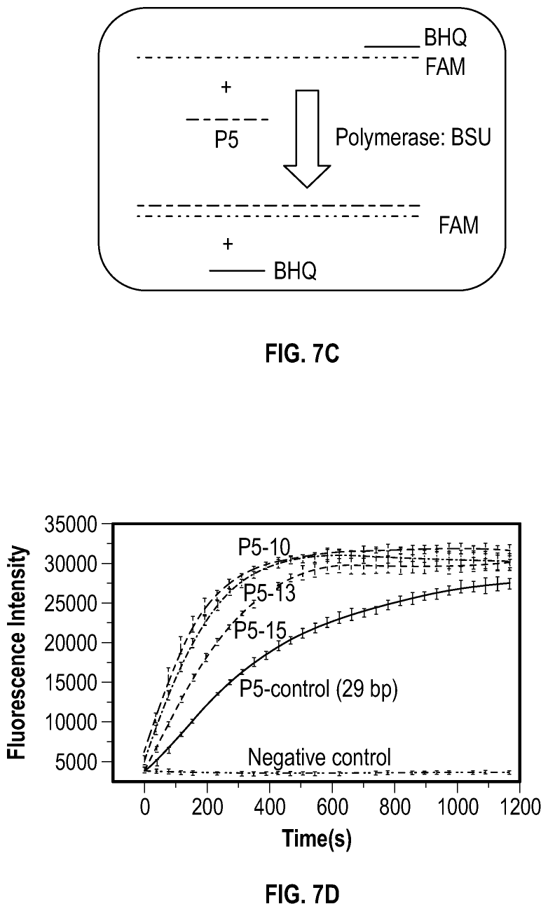

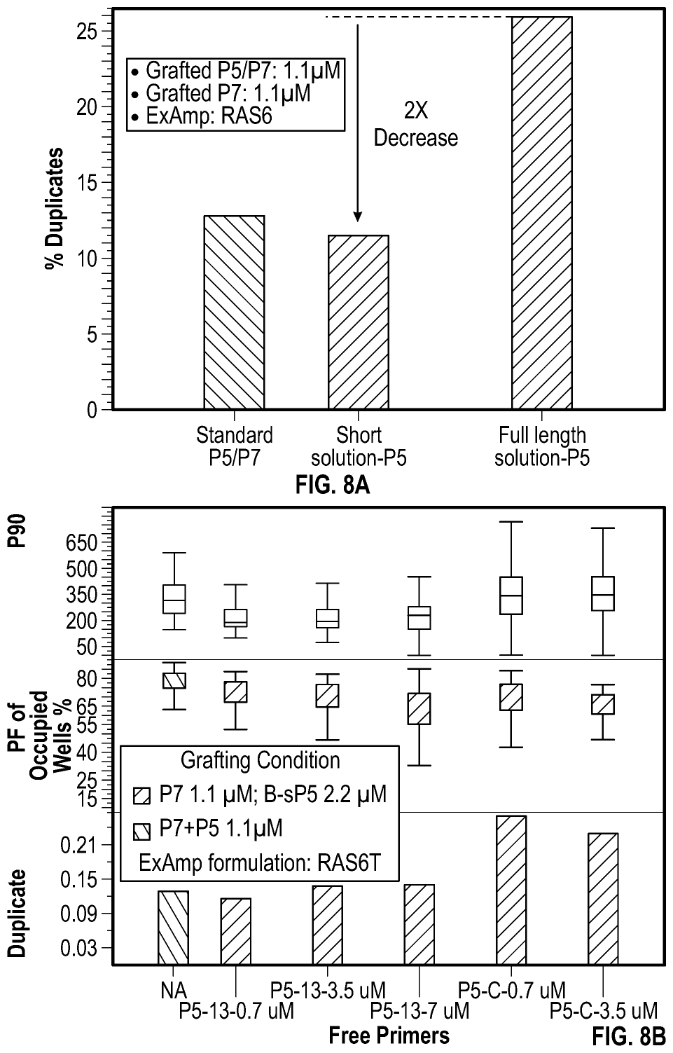

In one embodiment, the length of the solution-phase primers is between 5 and 25 bp or between 9 and 20 bp or between 5 and 15 bp or between 9 and 15 bp. In one embodiment, the length of the solution-phase primers is 10 bp, 13 bp or 15 bp. As above, the solution-phase primer may be a P5 or P7 primer. In one embodiment, the solution-phase primer is a P5 primer. In one embodiment, the solution phase primer is between 5 and 25 bp or between 10 and 20 bp or between 5 and 15 bp, preferably 10 bp, 13 bp or 15 bp of SEQ ID NO: 1 or 2. In other words, the solution-phase primer can be any—e.g. 13 bp of SEQ ID NO: 1 or 2. As shown in FIGS. 7A-7D solution-phase primers have lower rates of hybridisation and faster rates of hybridisation and extension compared to longer length primers (for example of 29 bp). As also shown in FIG. 8, solution-phase primers of this length are able to decrease the formation of duplicates by at least two-fold.

In addition, the resulting sequence performance (P90 and % PF) is comparable whether the smart solution primers of the disclosure or longer-length amplification primers are used. This is shown in FIG. 8B. Furthermore, as also shown in FIG. 8B, the amount of duplicates formed when smart solution primers are used is comparable to systems where full-length P5 and P7 lawn primers are used (compare the first bar with P5-13 bp of FIG. 8B).

In a further embodiment of the disclosure, the solution-phase primers comprise or consist of a nucleic acid sequence as defined in SEQ ID NO: 5, 6 or 7 or a variant thereof. In one embodiment, the solution-phase primers comprise or consist of SEQ ID NO: 6 or a variant thereof.

In another aspect of the disclosure, there is provided a solution-phase primer comprising or consisting of SEQ ID NO: 5, 6 or 7 or a variant thereof.

Following amplification of a template strand into a cluster, the next step in the process of sequencing the insert is sequencing of the forward strand and re-synthesis and sequencing of the reverse strand. In one embodiment this may be carried out by paired-end (PE) re-synthesis.

In one embodiment, PE re-synthesis is achieved using “blocked” or “dormant” lawn primers. These primers do not participate in cluster generation but only in re-synthesis prior to sequencing. In one embodiment, the lawn primer is blocked at the 3′ end, which is removed prior to re-synthesis—e.g. following generation of the cluster. In this way the lawn primer can be considered dormant until the sequencing step. The 3′ block may be a phosphate group or another reversible blocking group.

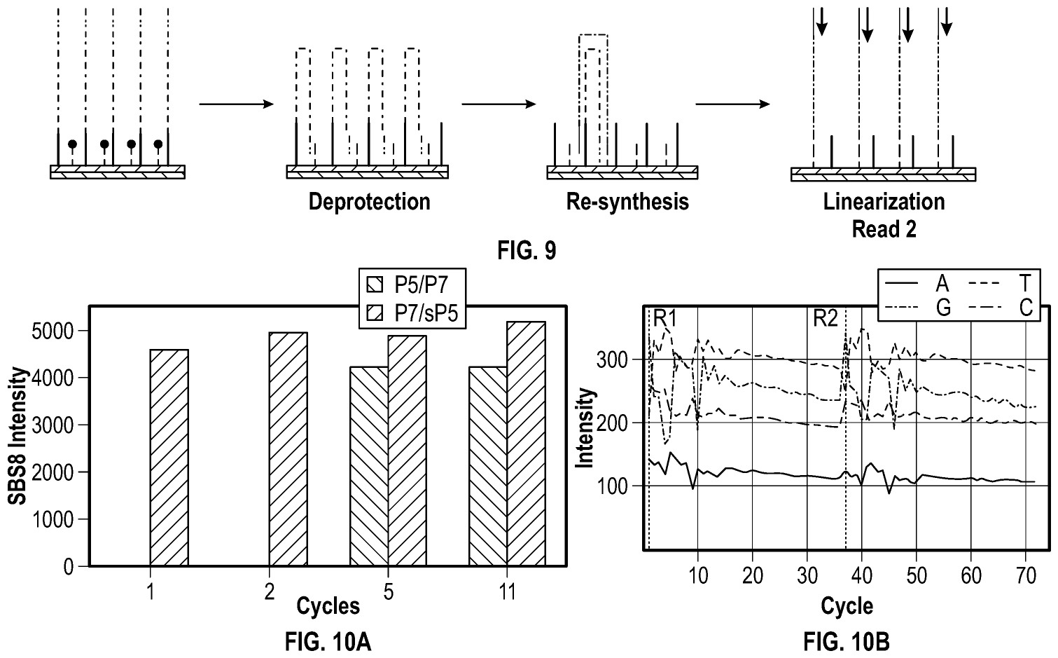

An exemplary method of sequencing according to the disclosure is shown in FIG. 2B and in FIG. 9. Following generation of the cluster (step 3 of FIG. 2B) all non-immobilised strands are removed from the surface. Where the lawn primer is P7 this means that all P5 strands (that is, strands comprising the P5 sequence as defined in SEQ ID NO: 1) are removed, leaving only P7 immobilised extended strands. The first sequencing read (R1) begins with binding and extension of the first sequencing primer (e.g. SBS3). Sequencing can be carried out using any suitable “sequencing-by-synthesis” technique as described above. In the next step, the dormant lawn primer (or “re-synthesis primer”) is unblocked, the immobilised extended strand bridges over (e.g. the P7 strand) providing a template for extension of the reverse strand (e.g. the P5 strand) from the now un-blocked dormant primer. The immobilised strand (i.e. the strand sequenced in R1) is removed and the now extended reverse strand linearized. The second sequencing primer binds and the second sequencing step (R2) can now proceed to sequence the reverse strand.

Accordingly, in a further aspect, the disclosure provides a method of sequencing a nucleic acid sequence, wherein the method comprises the following steps, as described above:
a. applying a nucleic acid template library in solution to a solid support; wherein the template library comprises a plurality of template strands, wherein each template strand comprises a first or second 5′ primer-binding sequence and a first or second 3′ primer binding sequence; and wherein the solid support has immobilised thereon a plurality of lawn primer sequences complementary to the 3′ primer-binding sequence; and a plurality of dormant lawn primers substantially complementary to the 3′ first or second primer-binding sequence, wherein the dormant lawn primers are blocked at the 3′end, and wherein the lawn and dormant lawn primers bind to different 3′-primer binding sequences;
b. hybridising the first or second 3′ primer binding sequence of the single stranded template strand to a first lawn primer;
c. carrying out an extension reaction to extend the lawn primer to generate a first immobilised strand complementary to the template strand, wherein the immobilised strand comprises a 3′ primer binding sequence;
d. displacing the template strand from the first immobilised strand and hybridising the single stranded template strand to a second lawn primer to provide said first single-stranded immobilised strand complementary to the template strand and a template strand hybridised to a second lawn primer;
e. providing a plurality of primers in solution, wherein the primers in solution are substantially complementary to the first or second 3′ primer binding sequences and hybridise to the 3′ end of the immobilised strand;
f. carrying out an extension reaction to extend the second lawn primer to generate a further immobilised stand, and the solution primer of step (e) to generate and a further template strand; and optionally
g. repeating steps (d) to (f) to produce a cluster of immobilised and template strands;
   and additionally,
h. selectively removing the non-immobilised template strands;
i. carrying out a first sequencing read to determine the sequence of a region of the immobilised strand; preferably by a sequencing-by-synthesis technique or by a sequencing-by ligation technique or a sequencing by hybridization technique;
j. selectively removing the sequencing product;
k. removing the blocking group from the dormant primers to allow hybridisation of the 3′ end of the immobilised strand to the unblocked primer;
l. carrying out an extension reaction to extend the unblocked primer using the immobilised strand as a template;
m. selectively removing the immobilised first sequencing read strand; and
n. carrying out a second sequencing read to determine the sequence of a region of the immobilised strand; preferably by a sequencing-by-synthesis technique or by a sequencing-by ligation technique.

In a further aspect, the disclosure provides a method of sequencing a target nucleic acid sequence, wherein the method comprises:
a. providing a solid support having immobilised thereon a cluster of first immobilised nucleic acid strands including said target nucleic acid sequence, wherein the solid support has a plurality of dormant lawn primers, wherein the dormant lawn primers are blocked at the 3′end;
b. carrying out a first sequencing read to determine the sequence of a region of the first immobilised strands; preferably by a sequencing-by-synthesis technique or by a sequencing-by ligation technique;
c. removing the blocking group from the dormant primers to allow hybridisation of a 3′ end of the first immobilised strand to the unblocked primer;
d. carrying out an extension reaction to extend the unblocked primer using the immobilised strand as a template to generate a cluster of second immobilised nucleic acid strands;
e. carrying out a second sequencing read to determine the sequence of a region of the second immobilised strand; preferably by a sequencing-by-synthesis technique or by a sequencing-by ligation technique;
   wherein determining the first and second sequences achieves pairwise sequencing of said target nucleic acid sequence.

Again, in one embodiment, the lawn primer may be a P5 primer and the dormant lawn primer may be a P7 primer. In another embodiment, the lawn primer may be a P7 primer and the dormant lawn primer a P5 primer. In other words, the lawn and the dormant lawn primers are different.

In one embodiment, the dormant lawn primer is a P5 primer and comprises or consists of a sequence as defined in SEQ ID NO: 8 or a variant thereof. This primer has a polyT provides spacer to reduce steric hindrance during the paired end turn re-synthesis. Shexynyl is a non-limiting example of a linking group that allows attachment of the primer to the surface of the sold support. Other linking groups would be apparent to the skilled person.

Paired-end re-synthesis in particular requires numerous cycles (11 in a standard cycle) because of surface P5 damage in the first linearization, where some of the P5 primers are not able to be extended. The damage can come from a possible incomplete chemical reaction (CCL1) or inaccurate enzyme (Uracil) catalysed cleavage. In the present disclosure, as only one type of lawn primers participate in generation of the cluster, the first linearization is not required in order to carry out read one (R1). Accordingly, the present disclosure provides a method of sequencing (e.g. by paired-end re-synthesis) that avoids damage to surface (i.e. lawn) primers (e.g. P5 lawn primers) during template amplification (i.e. cluster generation). This leads to more efficient PE re-synthesis. This is demonstrated by an increase in intensity of the second sequencing read (i.e. read 2) as shown in FIG. 10A. As further shown in FIGS. 10A-10B, the same level of signal intensity in the second sequencing read is also achieved using just 1 cycle, as shown in FIG. 10B. As such, the present disclosure also reduces the time needed to perform read 2, since a readable signal can be obtained with fewer cycles.

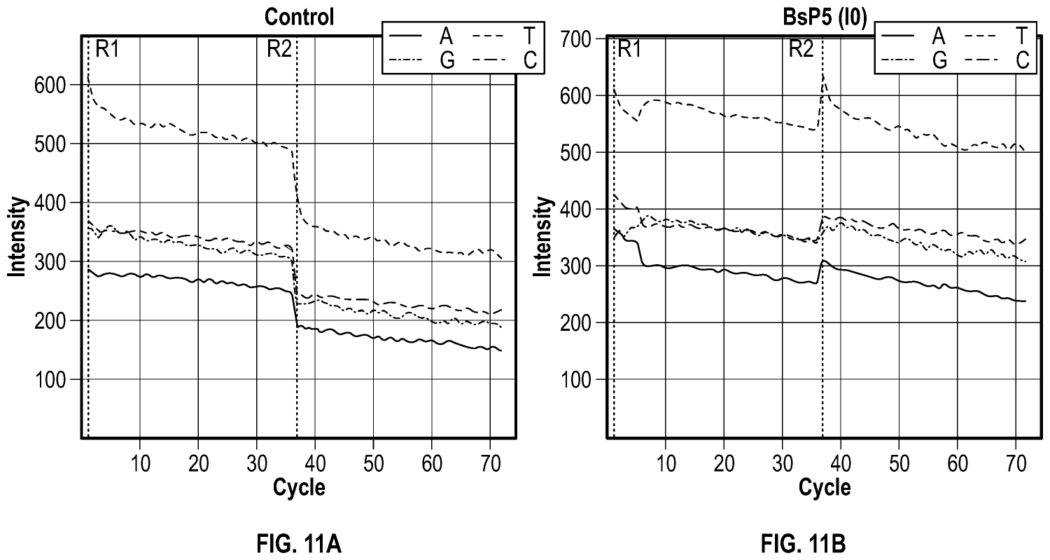

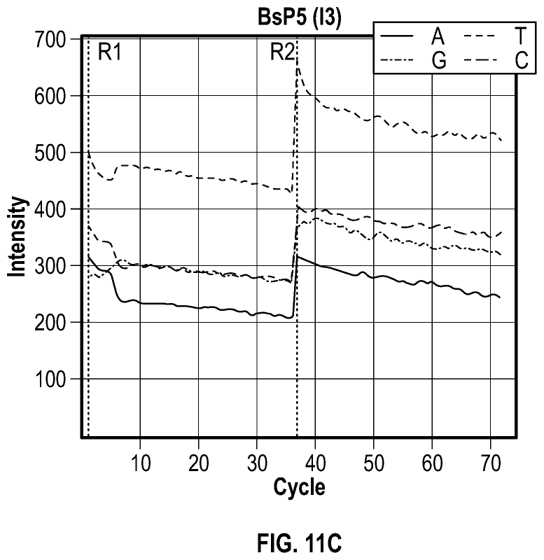

The increased efficiency of the present disclosure is further shown in FIGS. 11A-11C, which demonstrates that the use of non-bridging clustering leads to an improved signal intensity for read 2.

In one embodiment, the dormant lawn primer is grafted at a concentration in the range of 0.2 μM to 5 μM or 0.4 μM to 3 μM or 0.5 μM to 2.5 μM. In a further embodiment, the dormant lawn primer is grafted at 0.5, μM or 1.1 μM or 2.2 μM. In a preferred embodiment, the dormant lawn primer is grafted at 2.2 μM. The dormant lawn primer is either a P5 or P7 primer.

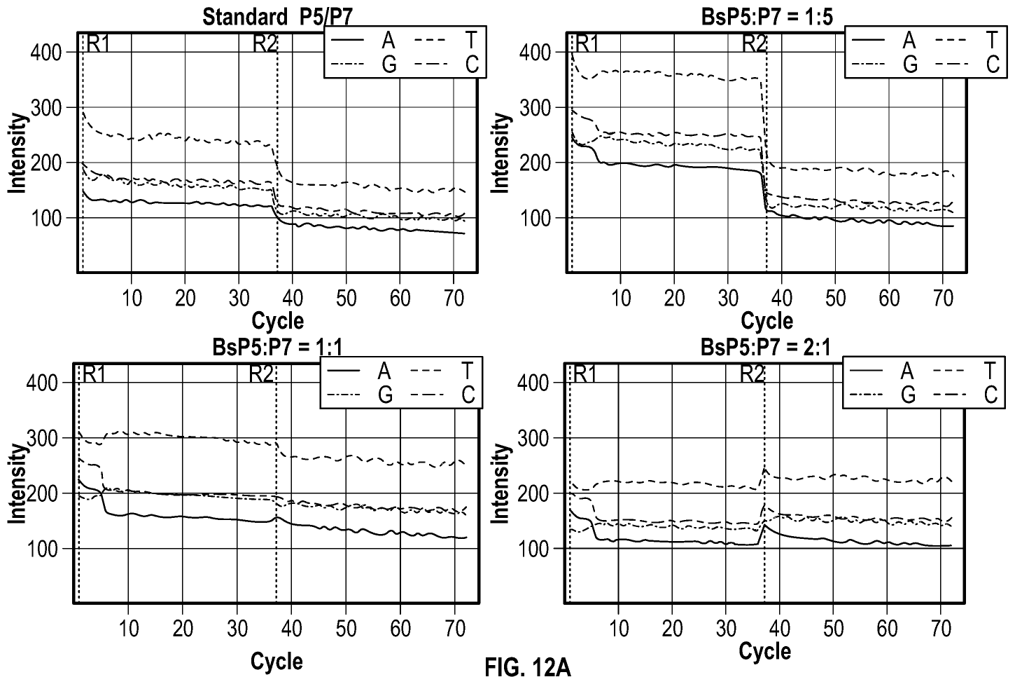

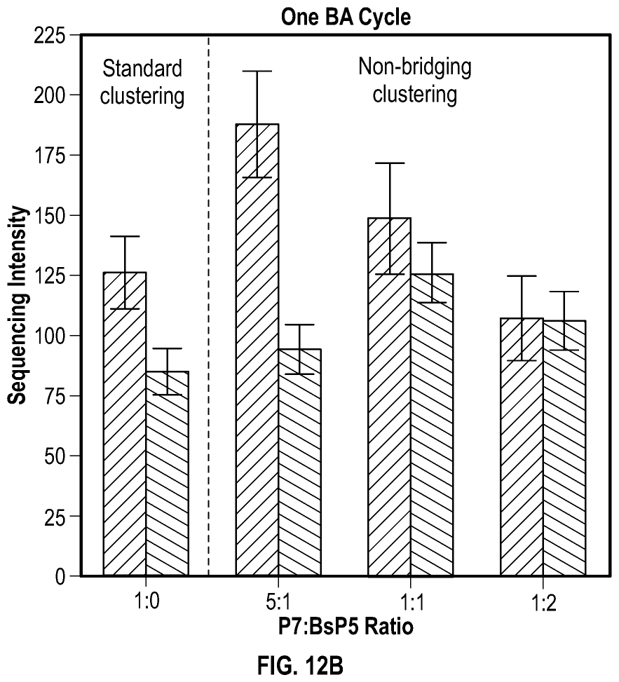

It has also been found that the ratio of lawn primers and dormant lawn primers affects read 1 and 2 intensity. As shown in FIGS. 12A-12B, a higher lawn: dormant lawn primer ratio (e.g. P7: BsP5) leads to a high R1 intensity but lower R2 intensity, while a lower lawn: dormant lawn primer ratio leads to a lower R1 intensity (compared to a higher lawn: dormant lawn primer ratio) but a higher R2 intensity. Accordingly, in one embodiment, the ratio of lawn:dormant lawn primer ratio is selected from 5:1, 4:1, 3:1, 2:1, 1:1 and 1:2, 1:3, 1:4 and 1:5. In a preferred embodiment, the ratio of lawn:dormant lawn primer ratio is selected from 2:1, 1:1 and 1:2.

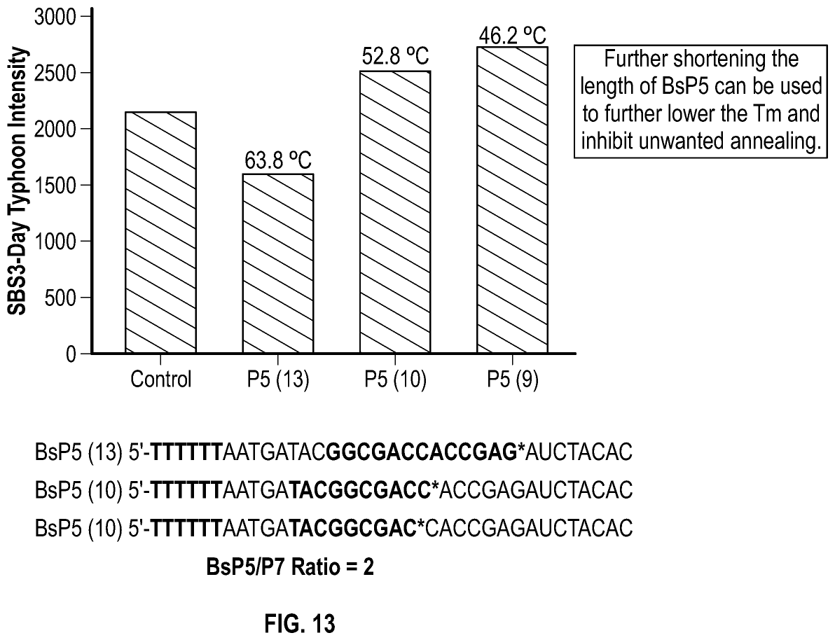

As the solution-phase primers are also shorter in length, in one embodiment, the dormant lawn primer may also be correspondingly shorter in length. In a further embodiment, the dormant lawn primer may also be between 5 and 25 bp or between 7 and 20 bp or between 9 and 13 bp. In one embodiment, the length of the dormant lawn primer is 9 bp, 10 bp or 13 bp. The use of shorter-length dormant primers, in addition to primers with a 3′ blocking group, not only prevents extension until following cluster generation but also prevents invasion (i.e. unwanted annealing), which would decrease amplification efficiency. As shown in FIG. 13 if the blocked short primer is too long the Read 1 signal intensity drops off in parallel with an increase in the Tm of the primers.

In a further embodiment, the dormant lawn primers may comprise or consist of a nucleic acid sequence selected from SEQ ID NO: 9, 10 or 11 or a variant thereof. The primers may also be blocked at the 3′ end (i.e. a 3′ blocking group), where the block prevents extension of the primer until the block is removed.

In a further aspect of the disclosure, there is provided a re-synthesis primer, the primer comprising a nucleic acid sequence selected from SEQ ID NO: 9, 10 or 11 or a variant thereof, and wherein the primer comprises a 3′ blocking group that prevents extension of the primer until the blocking group is removed. By “re-synthesis” is meant a primer that is capable of synthesising the reverse or complement strand after the first sequencing read (i.e. read 1). The re-synthesis primer is also referred to herein as a dormant lawn primer, and such terms may be used interchangeably.

In one embodiment the blocking group is a phosphate group. In one embodiment the surface of the solid support is treated with a phosphatase to remove the block.

In another aspect of the disclosure there is provided a solid support for use in sequencing, wherein the support comprises a plurality of lawn primers immobilised thereon and a plurality of dormant lawn primers immobilised thereon, wherein the dormant lawn primers comprise a blocking 3′ group that prevents extension until removed.

In one embodiment, the lawn primer is selected from a P7 or a P5 primer.

In another embodiment, the dormant lawn primer is selected from a P5 or a P7 primer. In a further embodiment, the dormant lawn primer comprises or consists of a nucleic acid sequence as defined in SEQ ID NO: 8, 9, 10 or 11 or a variant thereof.

In one embodiment, the ratio of lawn:dormant lawn primer ratio is selected from 5:1, 4:1, 3:1, 2:1, 1:1 and 1:2, 1:3, 1:4 and 1:5. In a preferred embodiment, the ratio of lawn:dormant lawn primer ratio is selected from 2:1, 1:1 and 1:2.

In further embodiments, the solid support does not require dormant lawn primers to achieve PE re-synthesis. Such a strategy is possible where bridge re-synthesis is not required to enable the second read to take place. An example is a system whereby two pads containing their own set of unique primers and complementary linearization chemistry (one set for read 1 and one set for read 2) are provided. An example of this strategy is using PAZAM pads as described in WO 2020/005503, the entire contents of which are incorporated by reference herein. In such an embodiment, the present disclosure can utilise the primer in solution approach of the present disclosure which avoids/minimises invasion and duplicate formation but does not require dormant lawn primers as described above since it is not necessary to undertake paired-end resynthesis.

The disclosure is now described in the following non-limiting examples:

### Example 1: Proof-of-Principle Hybrid Clustering

The disclosure is a new hybrid clustering methodology (as shown in FIGS. 2A-2D), that improves and addresses limitations of the current clustering strategies. In one example, the hybrid clustering approach employs both lawn (P7) and free solution primers (P5) for DNA amplification with paired-end (PE) sequencing ability, resulting in less steric hindrance and higher amplification flexibility, as well as non-bridged morphology of the DNA cluster. Also, the highlight of the hybrid clustering is the designed “smart” free solution-phase primers (P5), which can only hybridize and extend, with no invasion capability. Therefore, it would prevent extra duplicates from strand reseeding caused by the invasion of the solution P5.

To demonstrate the effectiveness of the present method, hybrid clustering performance has been evaluated through investigation of kinetics and cluster intensity. This experiment addresses the concern that flexible solution primers could generate primer dimers, which would influence the final sequencing intensity.

A wide range of concentrations titration on solution primers (P5) has been conducted with surface primers grafting at 1.1 μM. Real-time kinetics plot uses subtraction value of real-time intensity and initial intensity as the readout, as EvagreenR would vary the background signal corresponding to the amount of single strand DNA. However, hybrid clustering may not be accurately reflected if only relying on real-time EvaGreen intensity, as the significant background signal from the free solution primers. Thus, the investigation of clustering has been performed in combination of recording real-time intensity of EvaGreen and capturing final cluster intensity.

According to the result shown in FIG. 3B, a slight increase in cluster intensity was noticed along with elevation of the free solution primers' concentrations from 5 μM to 25 μM, followed by a decrease as concentration reaches a certain higher level (50 μM). This may be because of the formation of primer dimers resulting from an excessive amount of free solution primers. While the real-time kinetics (FIG. 3A) behaves similarly with free solution primers at lower concentration ranges, but as it falls in the higher concentration range, the kinetics curve cannot accurately capture the behaviours of clustering, as well as formation of primer dimers (orange lane is the control with no template seeding). This is probably due to variation in EvaGreen background signals. Moreover, as-designed hybrid clustering exhibit faster kinetics compared with current Illumina amplification strategy, as shown in FIG. 4. This study suggests that hybrid clustering is able to be used in amplification, where certain amount of free solution primers is required to achieve the optimized clustering performance.

### Example 2: Design of the “Smart” Solution Primer with No Invasion Competency

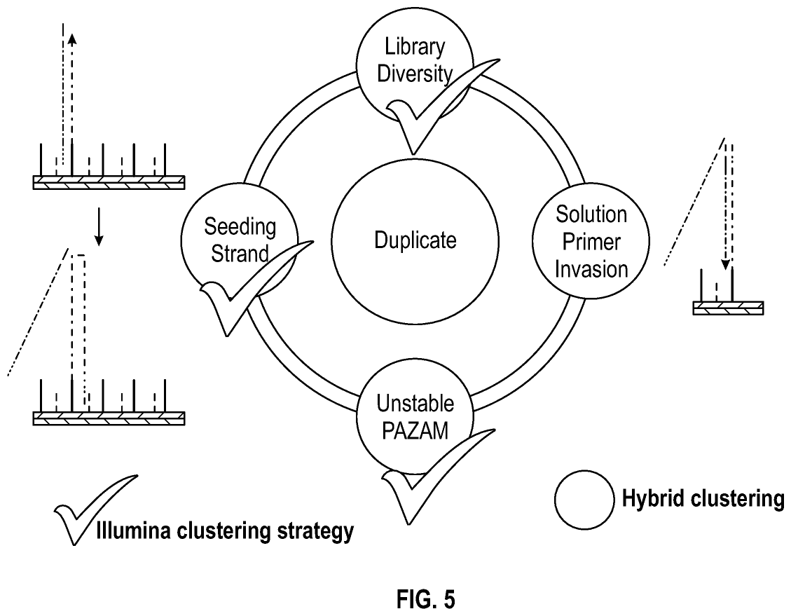

Percentage of duplicate reads is an important parameter in the evaluation of sequencing performance. Several factors can cause the generation of duplicate colonies as showing in FIG. 5. Some are due to system issues, such as library diversity (PCR duplicates), and re-seeding of free strands/tiny clusters along with unstable PAZAM layers on the flow cell. Some are ascribed to the re-seeding of the not anchored strands in both clustering strategies. In surface bridge clustering strategy, the initial extended copy strand can easily bridge over to the surface primers, leaving the free strands of the initial template. In the current hybrid clustering methodology, free strands would not be generated from the seeded template, but instead, result from the invasion of the solution primers. To avoid duplicates from the free strands' re-seeding, the hybrid clustering approach is designed with “smart” solution primers, which can only hyb/extend, but have reduced or no invasion capability (FIGS. 6A-6B).

In one embodiment, the clustering method is based on recombinase polymerase amplification (RPA) and it has been reported that the optimized length for RPA primers should be 30-35 bases long for the optimal formation of recombinase/primer filaments, with longer primers not being recommended. A hypothesis comes out that shortening the length of the primers may avoid the filament formation between recombinase and primer, and consequently lower or prevent invasion capability of the solution primers. Solution-based invasion and hyb/extension assays have been employed to test this hypothesis. Sequences of the primer at 10 (TACGGCGACC) (SEQ ID NO: 5), 13 (GGCGACCACCGAG) (SEQ ID NO: 6) and 15 (ACGGCGACCACCGAG) (SEQ ID NO: 7) bp length have been selected from 29 bp sequence of P5 primer. The scheme and the corresponding results of the invasion and hyb/extension of primers with different length are shown in the FIG. 7, demonstrating lower invasion and faster hyb/extension of the shorter solution primers.

For further validation, the sequencing performance has been evaluated. According to the result shown in FIGS. 8A-8B, hybrid clustering exhibits comparable values of P90 and PF as the normal bridging clustering strategy. The percentage of duplicate colonies of hybrid clustering decreases significantly with shorter solution P5 (sP5), reaching a value similar to the normal clustering strategy (surface P5/P7). Here the duplicates in the normal P5/P7 clustering is likely due to low diversity library and PAZAM-flake off, since there are no free library elements reseeding. The short P5 primers in solution have similar numbers of duplicates, which demonstrates that they are not making significant free templates for reseeding. Therefore, shorter P5 (13 bp) has been applied as “smart” solution primer for the hybrid clustering methodology.

Overall, to prevent invasion but maintain hyb/extension capability, the solution primers need to be designed to only form filament with polymerase, but not recombinase. Thus, besides tuning the length of the primers, a series of other possible approaches have been considered, such as modifying the backbone of the primers (decorating backbone with fluorine, incorporation of several PNA/LNA bases, internal mismatches sequence of the primers, or implementations with carbon spacers within primer sequence, etc), separately and in combination with modifications to the recombinase and or polymerase.

### Example 3: Design of Blocked Surface Primer for Faster PE Re-Synthesis

To obtain capability of PE sequencing, phosphate blocked P5 primers are grafted with surface clustering primer (P7) on the lawn. Surface-bounded blocked P5 is employed only for PE re-synthesis purpose, thus they are deprotected prior to PE turn. (scheme showing in the FIG. 9) In order to prevent the slowing down of ExAmp clustering inducing by the generation of filament between ExAmp and blocked P5, the short stumps of P5 were designed, which can be lengthened with a later hyb/extension step. As the solution-phase primers are also shorter length, corresponding blocked shorter P5 is designed with the following sequence (bold):

SEQ ID NO: 8)
/5Hexynyl/TTTTTTAATGATACGGCGACC
ACCGAG*A/ideoxyU/CTACAC

In this sense, the P5 lawn primers are ‘smart’ as well since they are designed to not only be blocked (preventing extension) but also be short enough to prevent invasion (non-productive) which could slow the ExAmp reaction (decreasing amplification efficiency).

PE re-synthesis efficiency was evaluated using hybrid clustering according to the present disclosure to quantify the effect of no surface P5 damage caused from the first linearization. PE re-synthesis test is firstly conducted by comparing the intensity of read 2 after different re-synthesis cycles (1, 2, 5, 11), where the normal Illumina clustering is carried out in parallel as the control experiment. The result suggests the hybrid clustering can achieve much higher read 2 intensity, and similar intensity under different re-synthesis cycles (blue bars in FIG. 10A). For further validation, a sequencing run using hybrid clustering has proved 1 cycle re-synthesis enables same R2 intensity as R1. (FIG. 10B) Therefore, as-designed hybrid clustering can also save time in PE re-synthesis.

SEQUENCE LISTING
SEQ ID NO: 1:
P5 sequence
AATGATACGGCGACCACCGAGATCTACAC
SEQ ID NO: 2:
P7 sequence
CAAGCAGAAGACGGCATACGAGAT
SEQ ID NO: 3
P5′ sequence (complementary to P5)
GTGTAGATCTCGGTGGTCGCCGTATCATT
SEQ ID NO: 4
P7′ sequence (complementary to P7)
ATCTCGTATGCCGTCTTCTGCTTG
SEQ ID NO: 5
short P5 primer
TACGGCGACC
SEQ ID NO: 6
short P5 primer
GGCGACCACCGAG
SEQ ID NO: 7
short P5 primer
ACGGCGACCACCGAG
SEQ ID NO: 8
/5Hexynyl/TTTTTTAATGATACGGC
GACCACCGAGA/ideoxyU/CTACAC
SEQ ID NO: 9
BsP5 (13)
TTTTTTGGCGACCACCGAG
SEQ ID NO: 10
BsP5(10)
TTTTTTTACGGCGACC
SEQ ID NO: 11
BsP5 (9)
TTTTTTTACGGCG

What is claimed is:

1. A method of amplifying a nucleic acid template, wherein the method comprises:
a. applying a nucleic acid template library in solution to a solid support; wherein the template library comprises a plurality of template strands, wherein each template strand comprises a first or second 5′ primer-binding sequence and a first or second 3′ primer binding sequence; and wherein the solid support has immobilised thereon a plurality of lawn primer sequences complementary to the 3′ primer-binding sequence;
b. hybridising the first or second 3′ primer binding sequence of the single stranded template strand to a first lawn primer;
c. carrying out an extension reaction to extend the lawn primer to generate a first immobilised strand complementary to the template strand, wherein the immobilised strand comprises a 3′ primer binding sequence;
d. displacing the template strand from the first immobilised strand and hybridising the single stranded template strand to a second lawn primer to provide said first single-stranded immobilised strand complementary to the template strand and a template strand hybridised to a second lawn primer;
e. providing a plurality of primers in solution, wherein the primers in solution are substantially complementary to the first or second 3′ primer binding sequences and hybridise to the 3′ end of the immobilised strand;
f. carrying out an extension reaction to extend the second lawn primer to generate a further immobilised stand, and the solution primer of step (e) to generate and a further template strand; and optionally
g. repeating steps (d) to (f) to produce a cluster of immobilised and template strands.

2. A method of sequencing a nucleic acid sequence, wherein the method comprises:
a. applying a nucleic acid template library in solution to a solid support; wherein the template library comprises a plurality of template strands, wherein each template strand comprises a first or second 5′ primer-binding sequence and a first or second 3′ primer binding sequence; and wherein the solid support has immobilised thereon a plurality of lawn primer sequences complementary to the 3′ primer-binding sequence; and a plurality of dormant lawn primers substantially complementary to the 3′ first or second primer-binding sequence, wherein the dormant lawn primers are blocked at the 3′end, and wherein the lawn and dormant lawn primers bind to different 3′-primer binding sequences;
b. hybridising the first or second 3′ primer binding sequence of the single stranded template strand to a first lawn primer;
c. carrying out an extension reaction to extend the lawn primer to generate a first immobilised strand complementary to the template strand, wherein the immobilised strand comprises a 3′ primer binding sequence;
d. displacing the template strand from the first immobilised strand and hybridising the single stranded template strand to a second lawn primer to provide said first single-stranded immobilised strand complementary to the template strand and a template strand hybridised to a second lawn primer;
e. providing a plurality of primers in solution, wherein the primers in solution are substantially complementary to the first or second 3′ primer binding sequences and hybridise to the 3′ end of the immobilised strand;
f. carrying out an extension reaction to extend the second lawn primer to generate a further immobilised stand, and the solution primer of step (e) to generate and a further template strand; and optionally
g. repeating steps (d) to (f) to produce a cluster of immobilised and template strands;
and additionally,
h. selectively removing the non-immobilised template strands;
i. carrying out a first sequencing read to determine the sequence of a region of the immobilised strand; preferably by a sequencing-by-synthesis technique or by a sequencing-by ligation technique;
j. selectively removing the sequencing product;
k. removing the blocking group from the dormant primers to allow hybridisation of the 3′ end of the immobilised strand to the unblocked primer;
l. carrying out an extension reaction to extend the unblocked primer using the immobilised strand as a template;
m. selectively removing the immobilised first sequencing read strand; and
n. carrying out a second sequencing read to determine the sequence of a region of the immobilised strand; preferably by a sequencing-by-synthesis technique or by a sequencing-by ligation technique.

3. The method of claim 1 or 2, wherein the first and second 3′ primer binding sequences are selected from P5′ and P7′, wherein P5′ comprises a nucleic acid sequence as defined in SEQ ID NO: 3 and wherein P7′ comprises a nucleic acid sequence as defined in SEQ ID NO: 4 or a variant thereof.

4. The method of any of claims 1 to 3, wherein the lawn primer is a P5 or P7 primer, wherein P5 comprises a nucleic acid sequence as defined in SEQ ID NO: 1 or a variant thereof and wherein P7 comprises a nucleic acid sequence as defined in SEQ ID NO: 2 or a variant thereof.

5. The method of any of claims 1 to 3, wherein the primer in solution is a P5 or P7 primer, wherein P5 comprises a nucleic acid sequence as defined in SEQ ID NO: 1 or a fragment thereof and wherein P7 comprises a nucleic acid sequence as defined in SEQ ID NO: 2 or a fragment thereof.

6. The method of any preceding claim, wherein the lawn primer is a P7 primer and the solution primer is a P5 primer.

7. The method of any of claims 1 to 5, wherein the lawn primer is a P5 primer and the solution primer is a P7 primer.

8. The method of any of claims 5 to 7, wherein the fragment is between 5 and 25 bp.

9. The method of claim 8, wherein the fragment is 10 bp, 13 bp or 15 bp.

10. The method of claim 9, wherein the primer comprises a nucleic acid sequence as defined in any of SEQ ID NO: 5, 6 or 7 or variants thereof.

11. The method of any of claims 2 to 10, wherein the dormant lawn primer is selected from a P5 and a P7 primer, wherein P5 comprises a nucleic acid sequence as defined in SEQ ID NO: 1 or a fragment thereof and wherein P7 comprises a nucleic acid sequence as defined in SEQ ID NO: 2 or a fragment thereof.

12. The method of claim 11, wherein the lawn primer is a P7 primer and the dormant lawn primer is a P5 primer.

13. The method of claim 11, wherein the lawn primer is a P5 primer and the dormant lawn primer is a P7 primer.

14. The method of any of claims 11 to 13, wherein the fragment is between 5 and 25 bp.

15. The method of claim 14, wherein the fragment is 9 bp, 10 bp or 13 bp.

16. The method of claim 15, wherein the dormant lawn primer comprises a nucleic acid sequence as defined in SEQ ID NO: 9, 10 or 11 or a variant thereof.

17. The method of any of claims 2 to 16, wherein the dormant lawn primer are blocked at the 3′ end by a phosphate group.

18. The method of claim 17, wherein the block is removed prior to step (1) by a phosphatase.

19. The method of any of claims 2 to 18, wherein the ration of lawn: dormant lawn primers is selected from 5:1, 4:1, 3:1, 2:1, 1:1 and 1:2, 1:3, 1:4 and 1:5.

20. A method of sequencing a target nucleic acid sequence, wherein the method comprises:
a. providing a solid support having immobilised thereon a cluster of first immobilised nucleic acid strands including said target nucleic acid sequence, wherein the solid support has a plurality of dormant lawn primers, wherein the dormant lawn primers are blocked at the 3′end;
b. carrying out a first sequencing read to determine the sequence of a region of the first immobilised strands; preferably by a sequencing-by-synthesis technique or by a sequencing-by ligation technique;
c. removing the blocking group from the dormant primers to allow hybridisation of a 3′ end of the first immobilised strand to the unblocked primer;
d. carrying out an extension reaction to extend the unblocked primer using the immobilised strand as a template to generate a cluster of second immobilised nucleic acid strands;
e. carrying out a second sequencing read to determine the sequence of a region of the second immobilised strand; preferably by a sequencing-by-synthesis technique or by a sequencing-by ligation technique;
wherein determining the first and second sequences achieves pairwise sequencing of said target nucleic acid sequence.

21. A solution-phase primer comprising or consisting of a nucleic acid sequence as defined in SEQ ID NO: 5, 6 or 7 or a variant thereof.

22. A re-synthesis primer, the primer comprising a nucleic acid sequence selected from SEQ ID NO: 9, 10 or 11 or a variant thereof, wherein the primer is blocked at the 3′ end to prevent extension of the primer until the block is removed.

23. The primer of claim 22, wherein the blocking group is a phosphate group.

24. A solid support for use in sequencing, wherein the support comprises a plurality of lawn primers immobilised thereon and a plurality of dormant lawn primers immobilised thereon, wherein the dormant lawn primers comprise a blocking 3′ group that prevents extension until removed.

25. The solid support of claim 24, wherein the lawn primer is selected from a P5 and a P7 primer, wherein P5 comprises a nucleic acid sequence as defined in SEQ ID NO: 1 or a variant thereof and wherein P7 comprises a nucleic acid sequence as defined in SEQ ID NO: 2 or a variant thereof.

26. The solid support of claim 24 or 25, wherein the dormant lawn primer is a P5 or P7 primer, wherein P5 comprises a nucleic acid sequence as defined in SEQ ID NO: 1 or a fragment thereof and wherein P7 comprises a nucleic acid sequence as defined in SEQ ID NO: 2 or a variant fragment thereof.

27. The solid support of any of claims 24 to 26, wherein the fragment is between 5 and 25 bp.

28. The solid support of claim 27, wherein the fragment is 9 bp, 10 bp or 13 bp.

29. The solid support of claim 28, wherein the dormant lawn primer comprises a nucleic acid sequence as defined in SEQ ID NO: 9, 10 or 11 or a variant thereof.

30. The solid support of any of claims 24 to 29, wherein the dormant lawn primers are blocked at the 3′ end by a phosphate group.

31. The solid support of any of claims 24 to 30, wherein the ratio of lawn:dormant lawn primer ratio is selected from 5:1, 4:1, 3:1, 2:1, 1:1 and 1:2, 1:3, 1:4 and 1:5.
